(19) United States
(12) Patent Application Publication
(10) Pub. No.: US 2011/0190243 A1
(43) Pub. Date: Aug. 4, 2011

(54) DIAGNOSIS AND TREATMENT OF BACK PAIN

(75) Inventor: Christopher M Bernards, Seattle, WA (US)

(73) Assignee: VIRGINIA MASON MEDICAL CENTER, Seattle, WA (US)

(21) Appl. No.: 13/121,859

(22) PCT Filed: Oct. 6, 2009
(86) PCT No.: PCT/US2009/059723

§ 371 Date: Mar. 30, 2011

Related U.S. Application Data
(60) Provisional application No. 61/103,075, filed on Oct. 6, 2008.

(51) Int. Cl. A61K 31/56; G01N 33/53; G01N 33/00; A61B 10/00

(52) U.S. Cl. 514/169; 435/7.92; 436/86; 600/562

(57) ABSTRACT

The present invention relates to methods of diagnosing and/or treating lower back pain (LBP). In one embodiment, the invention relates to a system for diagnosis of a form of lower back pain in an individual, using a means to identify a type and/or a concentration of one or more inflammatory mediators in the epidural space of the individual. In another embodiment, the invention relates to a system for diagnosis of a form of lower back pain in an individual by identifying a type and/or concentration of one or more inflammatory mediators in the epidural space of the individual, and rendering a diagnosis of the form of lower back pain in the individual based on the presence, absence and/or combination of presences and/or absences of one or more inflammatory mediators in the epidural space of the individual

### CROSS-REFERENCE TO RELATED APPLICATIONS

The present application claims the benefit of priority under 35 U.S.C. §119(e) of provisional application Ser. No. 61/103,075, filed Oct. 6, 2008, the contents of which are hereby incorporated by reference in their entirety.

### FIELD OF THE INVENTION

The invention relates generally to the fields of lumbar back pain/radiculopathy and inflammation and, more specifically, to the diagnosis and treatment of lower back pain.

### BACKGROUND

The following description includes information that may be useful in understanding the present invention. It is not an admission that any of the information provided herein is prior art or relevant to the presently claimed invention, or that any publication specifically or implicitly referenced is prior art.

Lumbar back pain/radiculopathy remains one of the most common problems for which patients seek medical attention. Unfortunately, modern understanding of the pathophysiology of back pain is rudimentary at best. Particularly perplexing is the occurrence of radicular pain in the absence of any evident anatomic basis (e.g., nerve compression) or pain that persists/worsens despite surgical removal of a seemingly clear anatomic cause (i.e., Failed Back Surgery Syndrome). One hypothesis that has been advanced to explain “non-anatomic” back pain is that it is caused by “inflammatory mediators” that activate local nociceptors, injure spinal nerves traversing the epidural space and cause central sensitization [13-17]. Examples of inflammatory mediators may include eicosanoids such as phospholipase A2 (PLA2), and cytokines such as IL-1 and TNFa.

Back pain/radiculopathy exacts a huge toll in human suffering and costs many billions of dollars annually in medical expenses and lost economic productivity. Thus, improving our understanding of the pathophysiology of this condition as well as developing new diagnostics and therapeutic approaches has the potential to pay huge dividends.

### SUMMARY OF THE INVENTION

Various embodiments include a method of diagnosing a form of lower back pain (LBP) in an individual, comprising obtaining a sample from the individual, assaying the sample to determine the type and/or concentration of one or more inflammatory mediators in the individual, and diagnosing the form of LBP based upon the type and/or concentration of one or more inflammatory mediators. In another embodiment, the sample is obtained from the epidural space of the individual. In another embodiment, the one or more inflammatory mediators comprise eicosanoids and/or cytokines. In another embodiment, the one or more inflammatory mediators comprise prostaglandin E2 (PGE2), leukotriene B4 (LTB4), thromboxane A2 (TXA2), tumor necrosis factor alpha (TNFa), interleukin-1 (IL-1), interleukin-6 (IL-6), and/or nitric oxide (NO). In another embodiment, the sample is obtained from the individual by microdialysis. In another embodiment, the sample is obtained from the individual using a CMA-71 High Cut-Off Brain Microdialysis Catheter. In another embodiment, the sample is assayed using a multiplexed assay kit. In another embodiment, the sample is assayed using ELISA and/or electrochemiluminescence. In another embodiment, the sample is assayed by measuring citruline concentrations. In another embodiment, the form of LBP comprises acute LBP, chronic LBP, clinical responsiveness to epidural steroids and/or responsiveness to drugs specifically targeting inflammatory mediators.

Other embodiments include a method for treating a form of lower back pain (LBP) in an individual, comprising identifying a type and/or concentration of one or more inflammatory mediators in the epidural space of the individual, rendering a diagnosis of the form of LBP in the individual based on the presence, absence and/or combination of presences and/or absences of one or more inflammatory mediators in the epidural space of the individual, and treating the form of LBP. In another embodiment, the one of the one or more inflammatory mediators in the epidural space of the individual comprises acute LBP, chronic LBP, clinical responsiveness to epidural steroids and/or responsiveness to drugs specifically targeting inflammatory mediators.

Other embodiments include a system for diagnosis of a form of lower back pain (LBP) in an individual, comprising a means to obtain a sample from the epidural space of the individual, and an assay to identify a type and/or a concentration of one or more inflammatory mediators from the sample. In another embodiment, the means to obtain a sample from the epidural space of the individual comprises microdialysis. In another embodiment, the microdialysis uses a CMA-71. High Cut-Off Brain Microdialysis catheter.

Other features and advantages of the invention will become apparent from the following detailed description, taken in conjunction with the accompanying drawings, which illustrate, by way of example, various embodiments of the invention.

### BRIEF DESCRIPTION OF THE FIGURES

FIG. 1 (prior art) depicts, in accordance with an embodiment herein, Meso Scale Discovery produces monoclonal antibodies (capture antibody) against the desired cytokines/eicosanoids and covalently attaches them to a carbon electrode plate. Samples are placed on the plate and the target eicosanoids/cytokines bind to the monoclonal antibody. A second monoclonal antibody to which an electrochemiluminescence tag has been covalently bound (labeled antibody) is added and it also binds to the cytokine/eicosanoid target molecule. Illumination of the sample produces a chemical reaction involving the label molecule, which generates electrons. The electrons produce a current the magnitude of which is proportional to the amount of labeled antibody, which in turn is proportional to the amount of cytokine/eicosanoid bound to the capture antibody.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

There is little doubt that low back pain (LBP) is one of the most common maladies experienced by the American public. Though epidemiological studies vary, the lifetime prevalence has been estimated to be as high as 60-85%. Not surprisingly, the direct medical cost for care of LBP is estimated to run into the billions of dollars each year in the United States. The indirect costs, which accrue from the fact that LBP is the leading cause of missed work, may run much higher. Apart from the considerable economic costs, the toll in human misery is staggering.

Despite the magnitude of the problem, LBP remains a poorly understood condition for which optimal therapy is unclear and, consequently, controversial. There are multiple reasons that LBP is poorly understood. Arguably the most important reason may be that “LBP” is a catch-all term that describes a single symptom that may result from “disease” originating from numerous anatomic sites (e.g., facet joints, paraspinous muscles, nerve roots, vertebral periosteum, etc.). In addition to variable anatomic underpinnings, LBP also varies in terms of its duration (e.g., acute, subacute, chronic). Thus, a huge number of potentially distinct “disease states” are lumped together as a single condition defined by the most prominent symptom: LBP. Consequently, our understanding of LBP and its treatment would be significantly advanced by identifying objective criteria that permit patients to be grouped according to common pathophysiologic mechanisms. The inventor's studies lay the groundwork for identifying biochemical markers of pathophysiologic mechanisms causing or contributing to LBP.

An important barrier to the accurate classification of LBP patients according to common mechanism is the difficulty of studying the pathophysiology of LBP clinically. In fact, the most common investigative tools employed in the diagnosis/treatment of LBP (i.e., MRI, myelogram, CT scan) are as likely to show normal anatomy or abnormalities incompatible with the patient's symptoms as they are to provide a clear anatomic etiology. Similarly, while nerve conduction studies may be helpful in indicating the presence of, or the location of, a neural abnormality they provide no pathophysiologic information.

Efforts to understand the pathophysiology of LBP in general, and specific patient's symptoms in particular, would benefit greatly from the ability to obtain diagnostic tissue or fluid, as is the case for so many other disease processes. Unfortunately, obtaining such samples was heretofore impractical. Microdialysis techniques have been used to study the pharmacology and physiology of the epidural and intrathecal environments in animal models [1-9] (and the inventor was the first to publish the use of microdialysis in the epidural space of animals for any purpose). In addition, the inventor has used in vivo microdialysis in the human peripheral nervous system to study local anesthetic pharmacokinetics and pharmacodynamics [10-12]. These studies raised the possibility of using microdialysis techniques in humans to obtain fluid samples to study the pathophysiology of LBP, but such studies were precluded by the lack of an appropriate microdialysis probe for use in the epidural space. However, such a probe is now approved for sampling the human brain and human epidural space (CMA-71 High Cut-Off Brain Microdialysis catheter, available from CMA Microdialysis, Inc., North Chelmsford, Mass.). The present invention is thus based, in part, on the use of this probe to investigate the role of inflammation in LBP. More specifically, the invention is based on the relationship between the type and concentration of inflammatory mediators (e.g., cytokines, eicosanoidss, nitric oxide) in the epidural space and the severity of LBP, the chronicity of LBP and the likelihood of response to epidural steroids or other more specific immune/inflammatory modulators (e.g., etanercept).

Thus, without wishing to be bound by any particular theory, the inventor believes that: (1) epidural microdialysis can be used to measure concentrations of pro-inflammatory cytokines (e.g., interleukins, chemokines, lymphokines), arachadonic acid metabolites (e.g. prostanoids, leukotrienes, thromboxanes), glutamate and nitric oxide (NO) in the epidural space of patients with acute and chronic LBP; (2) the types and concentrations of cytokines and arachadonic acid metabolites and the concentration of NO in the epidural space differ between patients with chronic LBP and those with acute LBP with radiculopathy; (3) elevated concentrations of arachadonic acid metabolites correlate with the clinical response to epidural steroids; and (4) elevated concentrations of nitric oxide NO, cytokines, and glutamate correlate with clinical response to drugs that specifically target these inflammatory mediators.

Therefore, in an embodiment, the invention relates to a system for diagnosis of a form of lower back pain in an individual, using a means to identify a type and/or a concentration of one or more inflammatory mediators in the epidural space of the individual. The means may be a CMA-71 High Cut-Off Brain Microdialysis catheter, available from CMA Microdialysis, Inc., North Chelmsford, Mass. or any similar device currently available or hereafter developed, which, in each case, enables one to identify a type and/or concentration of one or more inflammatory mediators in the epidural space of an individual. In another embodiment, the invention relates to a system for diagnosis of a form of lower back pain in an individual by identifying a type and/or concentration of one or more inflammatory mediators in the epidural space of the individual, and rendering a diagnosis of the form of lower back pain in the individual based on the presence, absence and/or combination of presences and/or absences of one or more inflammatory mediators in the epidural space of the individual. In another embodiment, the invention relates to a method for treating a form of lower back pain in an individual by identifying a type and/or concentration of one or more inflammatory mediators in the epidural space of the individual, rendering a diagnosis of the form of lower back pain in the individual based on the presence, absence and/or combination of presences and/or absences of one or more inflammatory mediators in the epidural space of the individual, and treating the form of lower pack pain.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Why Sample the Epidural Space

An assumption underlying the inventor's study design is that the epidural space is an appropriate site from which to obtain samples for analysis of inflammatory mediators contributing to LBP/radiculopathy. While not wishing to be bound by any particular theory, the inventor believes that this is a valid assumption for several reasons. As discussed above, animal models attempting to study the behavioral and histological effects of nucleus pulposus and individual inflammatory mediators have universally administered them into the epidural space and have found that this produces pain behaviors and histological changes that are consistent with the human clinical picture. Too, both animal studies and human in vitro tissue culture have identified intervertebral disc material as a source for the eicosanoids, cytokines, and NO thought to cause pain and neuropathy. In fact, disc material that herniated into the epidural space and was subsequently removed at surgery was the sole source for human tissue shown to produce inflammatory mediators in culture. Thus, inflammatory mediators produced by discs (actually by inflammatory cells present in diseased discs) are believed to be present in the epidural space. Finally, inflammatory mediators that might be produced at other peri-vertebral sites must traverse the epidural space to reach spinal nerve roots wherein they cause radiculopathy. Consequently, the epidural space is believed to be an appropriate site to obtain samples for analysis of inflammatory mediators.

### Example 2

### Study Population

A study includes three groups of subjects. One group (n=10) is patients with acute (less than 45 days duration) back pain and radiculopathy scheduled for epidural steroid injection. An additional inclusion criterion is that patients have had a lumbar spine MRI as part of their work-up and have no evidence of nerve root compression to explain their radicular symptoms. Finally, subjects must not have been on corticosteroids for any reason during the preceding 6 months.

The second group (n=10) is patients with chronic (greater than 12 months), axial back pain without radiculopathy.

The third group (n=10) is normal volunteers without a history of back pain.

The first two groups of patients are believed to likely represent two distinct pathologies (or at least different ends of a spectrum) and are, therefore, likely to demonstrate distinct patterns of inflammatory mediators (in terms of type and/or concentration). Thus, these two clinically different groups are appropriate to address all three of the specific aims of the research: (1) to test the hypothesis that eicosanoids, cytokines and NO can be measured in the epidural space of patients with acute and chronic LBP, (2) to test the hypothesis that the type and/or concentration of inflammatory mediators differs between patients with acute and chronic LBP, and (3) to test the hypothesis that the type and/or concentration of inflammatory mediators differs between patients who do, and who do not, experience a clinical benefit from epidural steroid injection. The control group addresses the question of whether the inflammatory mediators recovered from the epidural space of the two LBP groups are related to their LBP as opposed to a common finding in all humans.

It is believed that 30 patients are sufficient to detect any clinically significant differences between the two groups being studied. Subtle differences may not be statistically significant, but subtle correlations should be detectable as “trends.”

### Example 3

### MicroDialysis Probe

The microdialysis probe used in the study is a CMA-71 High Cut-Off Brain Microdialysis catheter, available from CMA Microdialysis, Inc., North Chelmsford, Mass. It is designed for the recovery of cytokines, which are too large to be recovered by “conventional” microdialysis probes. This probe also recovers smaller molecules like eicosanoids and citrulline (a surrogate for NO).

### Example 4

### MicroDialysis Procedure

Informed written consent is obtained from all subjects. In the acute LBP group, the epidural needle is placed in the lumbar epidural space at the level corresponding to the patient's radicular symptoms. In the chronic back pain group, the epidural needle is placed at the lumbar level thought to be responsible for generating the patient's symptoms. In both groups, the epidural needle is placed in the midline and advanced under fluoroscopic guidance using loss of resistance to 0.5 ml air as a means to help identify the epidural space. The loss of resistance to air technique is preferable to the saline loss of resistance technique for this study because the injected saline necessarily dilutes any inflammatory mediators present in the epidural space. Correct location of the needle tip is confirmed by successful passage of the microdialysis probe beyond the tip of the epidural needle and by visualizing the microdialysis probe within the epidural space (as opposed to exiting a neural foramen) using fluoroscopy. The microdialysis probe has a small gold tip to facilitate fluoroscopic imaging.

After placing the microdialysis probe, the epidural space is dialyzed for 30 minutes. The dialysate consists of saline containing 30 μg/ml dextran 60. Because of the high molecular weight cutoff (i.e. large pore size) of the dialysis probe, dextran 60 is necessary to prevent ultrafiltration of saline in this probe. The dialysate is pumped at 1 μl/min and collected into rubber-capped vials (CMA P000001 microvials) specially designed to handle low volumes and to prevent liquid evaporation. All samples are collected on water ice to prevent metabolism/breakdown of the recovered inflammatory mediators. The CMA model MD 107 pump is used to pump the dialysate through the microdialysis probe. This pump is specifically designed to accurately deliver the very slow infusion rates necessary for microdialysis and is FDA approved for human use. All samples are stored at −70° C. until thawed for analysis (see below).

Following the dialysis period, the microdialysis probe is removed. Subjects in the acute pain group then have a second epidural needle placed at the same level and receive their epidural steroid injection (80 mg methylprednisolone acetate).

### Example 5

### Data Collected

In addition to measuring the concentration of inflammatory mediators (see below), the following data is collected for subsequent correlation with the type and concentration of inflammatory mediators that are present:
1. Demographics: age, gender, height, weight, body mass index
2. Symptom duration:
3. Pain severity: 100 mm Visual Analog Scale. Although there is considerable debate as to the usefulness of the VAS as an assessment of back pain it has been shown to correlate with functional improvement following surgery for low back pain [43]. Consequently, the VAS is used in conjunction with questionnaires that use patient reports of functional capacity (see #4 below) to assess quantify severity of back pain and response to epidural steroids.
4. Patient Reported Functional Capacity: Both the Medical Outcomes Study Questionnaire Short Form 36 and the Oswestry Disability Index are administered to both groups on the day of microdialysis. These assessment tools were chosen because they have been shown to be sensitive indicators of improvement following both surgical and non-surgical treatments for back pain [35-43]. Both study groups repeat both assessments at 2, 4, 8, 16, and 26 weeks after microdialysis to assess for improvement. These data enable the determination as to whether any particular inflammatory mediator or threshold concentration of inflammatory mediator predicts either the magnitude or duration of response to epidural steroids.
5. Opioid use: Opioid use is converted to morphine equivalents using the Johns Hopkins Opioid Program conversion tables. A decrease in opioid usage serves as another indicator of response to epidural steroids.
6. Radiographic abnormalities: Lumbar spine MRI is used to determine whether radicular symptoms in the acute LBP group can be explained by spinal nerve compression. Patients who have evidence of spinal nerve compression that could explain their radicular symptoms are excluded from the study. All MRI's are read by a single radiologist blinded to the patient's group. The determination as to whether the patient's radicular symptoms can be explained by their lumbar spine MRI findings is made by a pain clinician in consultation with the radiologist who reads the MRI. This determination is made before the patient is enrolled in the study. The pain clinician is not otherwise involved in data collection or data analysis. The intent is to recruit subjects for this study for whom inflammatory mediators (as opposed to nerve compression) are a likely cause of their radicular symptoms. This approach may maximize the power of this study to detect an effect of inflammatory mediators as a correlate/cause of radiculopathy.
   This is not to suggest that inflammatory mediators may not be an important contributor to radiculopathy even in patients who do have evidence of spinal nerve compression. But, it is not possible to distinguish between compression effects and inflammatory mediator effects in a small pilot study if both are simultaneously present. However, the inventor anticipates that studies will address the relative contribution of compression and inflammation as causes of radiculopathy by identifying inflammatory mediators present in patients with nerve compression and specifically antagonizing them to determine whether this improves radiculopathy.
   Given that intervertebral disc pathology is believed to be the “agent” that initiates the inflammatory response quantified in this study, it is expected that many subjects (likely even control subjects) have some evidence of intervertebral disc pathology. This pathology is noted by the radiologist and reported. The MRI data is examined retrospectively with an eye toward determining whether any radiographic findings can be identified that correlate with the type/concentration of inflammatory mediators found. In fact, this analysis may contribute to identifying radiographic features that do, or do not, correlate with inflammation.
7. Inflammatory Mediators: Dialysates are assayed to determine concentrations of the following inflammatory mediators.
   i. PGE2
   ii. Leokotriene B4 (LTB4)
   iii. Thromboxane A2
   iv. TNFa
   v. Interleukin-1 (IL-1)
   vi. Interleukin-6 (IL-6)
   vii. NO (measured as citrulline, the stable byproduct of NO synthesis from arginine)

### Example 6

### Data Analysis

The exact approach to data analysis is ultimately determined by the nature of the data collected (e.g., parametric vs. non-parametric). But, in general terms correlations are identified between the types and amounts of inflammatory mediators present and whether the subjects' back pain is acute or chronic. In addition, correlations are identified between the types and concentrations of inflammatory mediators present and the response to epidural steroid injection (both magnitude and duration of response).

The variables being compared are not independent of one another. For example, opioid use, VAS, Oswestry disability index and Short-Form 36 are all interrelated measures of the same thing—patient discomfort. Similarly BMI, weight, and height are interdependent measures of body size. In addition, because the variables are all measured in the same subjects they are not independent of one another. The importance of the fact that these variables are not truly independent is that statistical power will not be markedly decreased by making multiple comparisons [46]. Consequently, it is not necessary to adjust P-values (e.g., Bonferonni correction) when determining statistical significance.

### Example 7

### Inflammatory Mediator Assays

Eicosanoid concentrations (PGE2, LTB4, TXA2) and cytokine concentrations (IL-1b, IL-6, TNFa) are analyzed using a custom multiplexed assay kit made specifically for us by Meso Scale Discovery (Gaithersburg, Md.). Meso Scale Discovery's technology successfully combines ELISA and electrochemiluminescence techniques to accurately detect less than 1 picogram amounts of cytokines/eicosanoids in sample volumes as low as 20 μl. This capability is useful for this study because microdialysis yields very small sample volumes −30 microliters in this study. The small sample volumes result from the slow dialysate flow rates that are necessary to permit equilibration of the dialysate with the tissue fluid in which the probe resides.

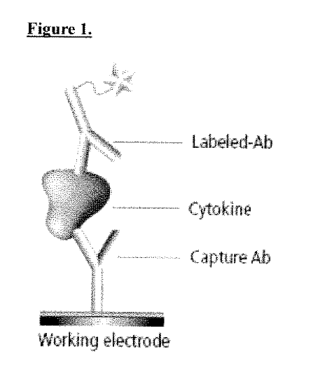

Briefly, as illustrated in FIG. 1, Meso Scale Discovery produces monoclonal antibodies (capture antibody) against the desired cytokines/eicosanoids and covalently attaches them to a carbon electrode plate. Samples are placed on the plate and the target eicosanoids/cytokines bind to the monoclonal antibody. A second monoclonal antibody to which an electrochemiluminescence tag has been covalently bound (labeled antibody) is added and it also binds to the cytokine/eicosanoid target molecule. Illumination of the sample produces a chemical reaction involving the label molecule, which generates electrons. The electrons produce a current the magnitude of which is proportional to the amount of labeled antibody, which in turn is proportional to the amount of cytokine/eicosanoid bound to the capture antibody. Up to 10 different compounds can be measured in a single well of a 96 well plate. Meso Scale Discovery produces individual kits designed to measure each of the eicosanoids/cytokines targeted in this study. A custom assay kit that simultaneously measures PGE2, TXA2, LTB4, IL-1b, IL-6 and TNFa in a single 20 microliter sample is produced specifically for the study. A Mesa Scale Discovery Sector 2400 imager is used to make measurements from the custom electrochemiluminescent antibody plate.

### Example 8

### Nitric Oxide Concentrations

Because NO is a gas, it is difficult to stably handle/store microdialysis samples containing NO without losing some of the dissolved gas. Consequently, investigators typically measure NO indirectly by measuring citrulline concentrations in the dialysate [47-50]. Citrulline is the amino acid byproduct of nitric oxide synthase-mediated generation of NO from L-arginine. NO and citrulline are produced in equi-molar concentrations, thus, citrulline concentration accurately reflects NO concentration in tissues.

Citrulline is measured using a 6-aminoquinolyl-N-hydroxysuccinimidyl carbonate (AQC) derivatization and a high performance liquid chromatography/mass spectroscopy assay modified from DeAntonis et al. [51], and Cohen et al. [52]. This assay was previously used by the inventor to measure citrulline concentrations in microdialysis samples obtained in animal studies of inflammatory mediators produced in response to spinal cord injury [2]. Briefly, the derivatizing reagent, 6-aminoquinolyl-N-hydroxysuccinimidyl carbamate (AQC), is synthesized according to the method of Cohen et al. [52] Dialysate samples are thawed on ice and 10 μl added to 12×75 mm glass tubes. Twenty microliters of internal standard (20 ng/μl phosphoserine in water) is added to each tube. Borate buffer (40 μl, 0.2M, pH 8.8) is added to each tube followed by 20 μl AQC reagent. The solution is mixed well and incubated at room temperature for 1 minute and then mixed again. The resultant solution is transferred to an autosampler vial and capped with a Teflon lined crimp cap. The solution is incubated at 55° C. for 10 minutes and allowed to cool to room temperature. Ten microliters of this solution is injected onto the HPLC.

The HPLC (Agilent Technologies series 1100, Palo Alto, Calif.) is fitted with a C18 column (Restek Allure model 9164572, Bellefonte, Pa.) and the mobile phase consists of 85% 20 mM ammonium acetate, 0.02% N, N-dimethylhexylamine (pH 5.5) and 15% acetronitrile at a flow rate of 0.250 ml/min from 0 to 5.2 minutes and then 0.3 ml/min until 8 minutes. Post-time flow is 0.250 ml/min for 1.5 minutes. The column temperature is 45° C. and the sample temperature 25° C.

The mass spectrometer (Agilent Technologies series 1100, Palo Alto, Calif.) is set in high-resolution mode using selective ion monitoring of ions 346 m/z (citrulline) and 356 m/z (phosphoserine internal standard). Fragmentor is 70 volts, gain 1 and dwell 294 msec. Nitrogen drying gas flow is 10 L/min at 350° C. Quadrulpole temperature is 100° C. Nebulizing gas is nitrogen at a pressure of 35 psig. Capillary voltage is 2500 V.

A standard curve is run for both amino acids (citrulline and phosphoserine) using standards prepared in water from commercially purchased amino acids (Sigma-Aldrich, St Louis, Mo.).

### Example 9

### Table 1—Examples of Cytokines as Possible Inflammatory Mediators

TABLE 1

| Cytokine | Producing Cell | Target Cell | Function |
|---|---|---|---|
| IL-1 | Monocytes | Th-cells | Co-stimulation |
| | Macrophages | B-cells | Proliferation |
| | B-cells | NK-cells | Activation |
| Il-2 | Th1 cells | T, B, NK cells | Proliferation, Activation |
| IL-3 | NK cells | Mast cells | Histamine release |
| IL-4 | Th2 cells | B cells | IgG, IgE Synthesis |
| | | T cells | Proliferation |
| IL-5 | Th2 cells | B cells | IgA Synthesis |
| IL-6 | Macrophages | Plasma cells | Antibody Secretion |
| | Stromal cells | Various | Acute Phase Response |
| IL-8 | Macrophages | Neutrophils | Chemotaxis |
| | Endothelial cells | | |
| IL-10 | Th2 cells | Macrophages | Cytokine Production |
| | | B cells | Activation |
| IL-12 | Macrophages | NK cells | Activation |
| MIP-1a | Macrophages | Monocytes, T cells | Chemotaxis |
| MIP 1b | Lymphocytes | Monocytes, T cells | Chemotaxis |
| TNFa | Macrophages | Macrophages | CAM and Cytokine Expression |
| | Mast cells, NK cells | | |
| TNFb | Th1, Tc cells | Phagocytes | Phagocytosis, NO Production |

IL = interleukin, Th = T-helper, NK = natural killer, MIP = macrophage inflammatory protein

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

1. Bernards C M: Cerebrospinal fluid and spinal cord distribution of baclofen and bupivacaine during slow intrathecal infusion in pigs. Anesthesiology 2006; 105: 169-78
2. Bernards C M, Akers T: Effect of postinjury intravenous or intrathecal methylprednisolone on spinal cord excitatory amino-acid release, nitric oxide generation, PGE(2) synthesis, and myeloperoxidase content in a pig model of acute spinal cord injury. Spinal Cord 2006
3. Bernards C M, Shen D D, Sterling E S, Adkins J E, Risler L, Phillips B, Ummenhofer W: Epidural, cerebrospinal fluid, and plasma pharmacokinetics of epidural opioids (part 2): effect of epinephrine. Anesthesiology 2003; 99: 466-75
4. Bernards C M, Shen D D, Sterling E S, Adkins J E, Risler L, Phillips B, Ummenhofer W: Epidural, cerebrospinal fluid, and plasma pharmacokinetics of epidural opioids (part 1): differences among opioids. Anesthesiology 2003; 99: 455-65
5. Bernards C M, Sorkin L S: Radicular artery blood flow does not redistribute fentanyl from the epidural space to the spinal cord. Anesthesiology 1994; 80: 872-8
6. Bethune C R, Bernards C M, Bui-Nguyen T, Shen D D, Ho R J: The role of drug-lipid interactions on the disposition of liposome-formulated opioid analgesics in vitro and in vivo. Anesth Analg 2001; 93: 928-33
7. Koszdin K L, Shen D D, Bernards C M: Spinal cord bioavailability of methylprednisolone after intravenous and intrathecal administration: the role of P-glycoprotein. Anesthesiology 2000; 92: 156-63
8. Krupp J L, Bernards C M: Pharmacokinetics of intrathecal oligodeoxynucleotides. Anesthesiology 2004; 100: 315-22
9. Ummenhofer W C, Arends R H, Shen D D, Bernards C M: Comparative spinal distribution and clearance kinetics of intrathecally administered morphine, fentanyl, alfentanil, and sufentanil. Anesthesiology 2000; 92: 739-53
10. Bernards C M, Kopacz D J: Effect of epinephrine on lidocaine clearance in vivo: a microdialysis study in humans. Anesthesiology 1999; 91: 962-8
11. Kopacz D J, Bernards C M: Effect of clonidine on lidocaine clearance in vivo: a microdialysis study in humans. Anesthesiology 2001; 95: 1371-6
12. Kopacz D J, Bernards C M, Allen H W, Landau C, Nandy P, Wu D, Lacouture P G: A model to evaluate the pharmacokinetic and pharmacodynamic variables of extended-release products using in vivo tissue microdialysis in humans: bupivacaine-loaded microcapsules. Anesth Analg 2003; 97: 124-31 table of contents
13. Ghilardi J R, Svensson C I, Rogers S D, Yaksh T L, Mantyh P W: Constitutive spinal cyclooxygenase-2 participates in the initiation of tissue injury-induced hyperalgesia. J Neurosci 2004; 24: 2727-32
14. Harvey R J, Depner U B, Wassle H, Ahmadi S, Heindl C, Reinold H, Smart T G, Harvey K, Schutz B, Abo-Salem O M, Zimmer A, Poisbeau P, Welzl H, Wolfer D P, Betz H, Zeilhofer H U, Muller U: GlyR alpha3: an essential target for spinal PGE2-mediated inflammatory pain sensitization. Science 2004; 304: 884-7
15. Ma W, Eisenach J C: Morphological and pharmacological evidence for the role of peripheral prostaglandins in the pathogenesis of neuropathic pain. Eur J Neurosci 2002; 15: 1037-47
16. Rutkowski M D, Winkelstein B A, Hickey W F, Pahl J L, DeLeo J A: Lumbar nerve root injury induces central nervous system neuroimmune activation and neuroinflammation in the rat: relationship to painful radiculopathy. Spine 2002; 27: 1604-13
17. Winkelstein B A: Mechanisms of central sensitization, neuroimmunology & injury biomechanics in persistent pain: implications for musculoskeletal disorders. J Electromyogr Kinesiol 2004; 14: 87-93
18. Franson R C, Saal J S, Saal J A: Human disc phospholipase A2 is inflammatory. Spine 1992; 17: S129-32
19. Saal J S: The role of inflammation in lumbar pain. Spine 1995; 20: 1821-7
20. Saal J S, Franson R C, Dobrow R, Saal J A, White A H, Goldthwaite N: High levels of inflammatory phospholipase A2 activity in lumbar disc herniations. Spine 1990; 15: 674-8
21. Kang J D, Georgescu H I, McIntyre-Larkin L, Stefanovic-Racic M, Donaldson W F, 3rd, Evans C H: Herniated lumbar intervertebral discs spontaneously produce matrix metalloproteinases, nitric oxide, interleukin-6, and prostaglandin E2. Spine 1996; 21: 271-7
22. Kang J D, Georgescu H I, McIntyre-Larkin L, Stefanovic-Racic M, Evans C H: Herniated cervical intervertebral discs spontaneously produce matrix metalloproteinases, nitric oxide, interleukin-6, and prostaglandin E2. Spine 1995; 20: 2373-8
23. Nygaard O P, Mellgren S I, Osterud B: The inflammatory properties of contained and noncontained lumbar disc herniation. Spine 1997; 22: 2484-8
24. Chen C, Cavanaugh J M, Ozaktay A C, Kallakuri S, King A I: Effects of phospholipase A2 on lumbar nerve root structure and function. Spine 1997; 22: 1057-64
25. Kawakami M, Tamaki T, Hayashi N, Hashizume H, Nishi H: Possible mechanism of painful radiculopathy in lumbar disc herniation. Clin Orthop Relat Res 1998: 241-51
26. Kawakami M, Matsumoto T, Tamaki T: Roles of thromboxane A2 and leukotriene B4 in radicular pain induced by herniated nucleus pulposus. J Orthop Res 2001; 19: 472-7
27. Carette S, Leclaire R, Marcoux S, Morin F, Blaise G A, St-Pierre A, Truchon R, Parent F, Levesque J, Bergeron V, Montminy P, Blanchette C: Epidural corticosteroid injections for sciatica due to herniated nucleus pulposus. N Engl J Med 1997; 336: 1634-40
28. Aoki Y, Rydevik B, Kikuchi S, Olmarker K: Local application of disc-related cytokines on spinal nerve roots. Spine 2002; 27: 1614-7
29. Olmarker K, Rydevik B: Selective inhibition of tumor necrosis factor-alpha prevents nucleus pulposus-induced thrombus formation, intraneural edema, and reduction of nerve conduction velocity: possible implications for future pharmacologic treatment strategies of sciatica. Spine 2001; 26: 863-9
30. Ozaktay A C, Kallakuri S, Takebayashi T, Cavanaugh J M, Asik I, DeLeo J A, Weinstein J N: Effects of interleukin-1 beta, interleukin-6, and tumor necrosis factor on sensitivity of dorsal root ganglion and peripheral receptive fields in rats. Eur Spine J 2006; 15: 1529-37
31. Le Maitre C L, Hoyland J A, Freemont A J: Catabolic cytokine expression in degenerate and herniated human intervertebral discs: IL-1beta and TNFalpha expression profile. Arthritis Res Ther 2007; 9: R77
32. Tobinick E, Davoodifar S: Efficacy of etanercept delivered by perispinal administration for chronic back and/or neck disc-related pain: a study of clinical observations in 143 patients. Curr Med Res Opin 2004; 20: 1075-85
33. Cohen S P, Wenzell D, Hurley R W, Kurihara C, Buckenmaier C C, 3rd, Griffith S, Larkin T M, Dahl E, Morlando B J: A double-blind, placebo-controlled, dose-response pilot study evaluating intradiscal etanercept in patients with chronic discogenic low back pain or lumbosacral radiculopathy. Anesthesiology 2007; 107: 99-105
34. Cohen S A, Bogduk N: A pilot study evaluating transforaminal etanercept for lumbosacral radiculopathy, International Spinal. Intervention Society. Las Vegas, 2008
35. Ahn S H, Cho Y W, Ahn M W, Jang S H, Sohn Y K, Kim H S: mRNA expression of cytokines and chemokines in herniated lumbar intervertebral discs. Spine 2002; 27: 911-7
36. Brisby H, Olmarker K, Larsson K, Nutu M, Rydevik B: Proinflammatory cytokines in cerebrospinal fluid and serum in patients with disc herniation and sciatica. Eur Spine J 2002; 11:62-6
37. Miyamoto H, Saura R, Harada T, Doha M, Mizuno K: The role of cyclooxygenase-2 and inflammatory cytokines in pain induction of herniated lumbar intervertebral disc. Kobe J. Med. Sci 2000; 46: 13-28
38. Tobinick E L, Britschgi-Davoodifar S: Perispinal TNF-alpha inhibition for discogenic pain. Swiss Med Wkly 2003; 133: 170-7
39. Brisby H, Ashley H, Diwan A D: In vivo measurement of facet joint nitric oxide in patients with chronic low back pain. Spine 2007; 32: 1488-92
40. Kim S F, Hurl D A, Snyder S H: Inducible nitric oxide synthase binds, S-nitrosylates, and activates cyclooxygenase-2. Science 2005; 310: 1966-70
41. Sautebin L, Ialenti A, Ianaro A, Di Rosa M: Relationship between nitric oxide and prostaglandins in carrageenin pleurisy. Biochem Pharmacol 1998; 55: 1113-7
42. Bernards C M: Cyclosporine-A-mediated inhibition of p-glycoprotein increases methylprednisolone entry into the central nervous system. Spinal Cord 2005
43. Copay A G, Glassman S D, Subach B R, Berven S, Schuler T C, Carreon L Y: The minimum clinically important difference in lumbar spine surgery patients: a choice of methods using the Oswestry Disability Index, Medical Outcomes Study questionnaire Short Form 36, and Pain Scales. Spine J 2008
44. Ferrari R: Responsiveness of the short-form 36 and oswestry disability questionnaire in chronic nonspecific low back and lower limb pain treated with customized foot orthotics. J Manipulative Physiol Ther 2007; 30: 456-8
45. Veresciagina K, Ambrozaitis K V, Spakauskas B: Health-related quality-of-life assessment in patients with low back pain using SF-36 questionnaire. Medicina (Kaunas) 2007; 43: 607-13
46. Bland M: Multiple Significance Tests, An introduction to Medical Statistics. Oxford, Oxford University Press, 1989, pp 148-162
47. Engelmann M, Wolf G, Putzke J, Bloom F E, Raber J, Landgraf R, Spina M G, Horn T F: Nitric oxide is not involved in the control of vasopressin release during acute forced swimming in rats. Amino Acids 2004; 26: 37-43
48. Saul'skaya N B, Savel'ev S A, Solov'eva N A. Fofonova N V: NO synthase-dependent increases in extracellular citrulline levels in the nucleus accumbens in an emotional conditioned reflex. Neurosci Behav Physiol 2007; 37: 803-9
49. Savel'ev S A, Saul'skaya N B: Extracellular citrulline levels in the nucleus accumbens during the acquisition and extinction of a classical conditioned reflex with pain reinforcement. Neurosci Behav Physiol 2007; 37: 249-56
50. Van Hemelrijck A, Hachimi-Idrissi S, Sarre S, Ebinger G, Michotte Y: Post-ischaemic mild hypothermia inhibits apoptosis in the penumbral region by reducing neuronal nitric oxide synthase activity and thereby preventing endothelin-1-induced hydroxyl radical formation. Eur J Neurosci 2005; 22: 1327-37
51. De Antonis K M, Brown P R, Cohen S A: High-performance liquid chromatographic analysis of synthetic peptides using derivatization with 6-aminoquinolyl-N-hydroxysuccinimidyl carbamate. Anal Biochem 1994; 223: 191-7
52. Cohen S A, Michaud D P: Synthesis of a fluorescent derivatizing reagent, 6-aminoquinolyl-N-hydroxysuccinimidyl carbamate, and its application for the analysis of hydrolysate amino acids via high-performance liquid chromatography. Anal Biochem 1993; 211: 279-87
53. Inman S L, Faut-Callahan M, Swanson B A, Fillingim R B: Sex differences in responses to epidural steroid injection for low back pain. J Pain 2004; 5: 450-7
54. LaCroix-Fralish M L, Rutkowski M D, Weinstein J N, Mogil J S, Delco J A: The magnitude of mechanical allodynia in a rodent model of lumbar radiculopathy is dependent on strain and sex. Spine 2005; 30: 1821-7
55. Robinson M E, Dannecker E A, George S Z, Otis J, Atchison J W, Fillingim R B: Sex differences in the associations among psychological factors and pain report: a novel psychophysical study of patients with chronic low back pain. J Pain 2005; 6: 463-70
56. Marsala M, Malmberg A B, Yaksh T L: The spinal loop dialysis catheter: characterization of use in the unanesthetized rat. J Neurosci Methods 1995; 62: 43-53
57. Romero S D, Chyatte D, Byer D E, Romero J C, Yaksh T L: Measurement of prostaglandins in the cerebrospinal fluid in cat, dog, and man. J Neurochem 1984; 43: 1642-9
58. Yaksh T L, Horais K A, Tozier N, Rathbun M, Richter P, Rossi S, Grafe M, Tong C, Meschter C, Cline J M, Eisenach J: Intrathecal ketorolac in dogs and rats. Toxicol Sci 2004; 80: 322-34
59. Ghersi-Egea J F, Leininger-Muller B, Cecchelli R, Fenstermacher J D: Blood-brain interfaces: relevance to cerebral drug metabolism. Toxicol Lett 1995; 82-83: 645-53
60. Ghersi-Egea J F, Leninger-Muller B, Suleman G, Siest G, Minn A: Localization of drug-metabolizing enzyme activities to blood-brain interfaces and circumventricular organs. J Neurochem 1994; 62: 1089-96
61. Kern C, Bernards C M: Ascorbic acid inhibits spinal meningeal catechol-o-methyl transferase in vitro, markedly increasing epinephrine bioavailability. Anesthesiology 1997; 86: 405-9
62. Kern C, Mautz D S, Bernards C M: Epinephrine is metabolized by the spinal meninges of monkeys and pigs. Anesthesiology 1995; 83: 1078-81
63. Morse D C, Stein A P, Thomas P E, Lowndes H E: Distribution and induction of cytochrome P450 1A1 and 1A2 in rat brain. Toxicol Appl Pharmacol 1998; 152: 232-9
64. Okada T, Mochizuki T, Huang Z L, Eguchi N, Sugita Y, Urade Y, Hayaishi O: Dominant localization of adenosine deaminase in leptomeninges and involvement of the enzyme in sleep. Biochem Biophys Res Commun 2003; 312: 29-34
65. Ummenhofer W C, Brown S M, Bernards C M: Acetylcholinesterase and butyrylcholinesterase are expressed in the spinal meninges of monkeys and pigs. Anesthesiology 1998; 88: 1259-65

What is claimed is:

1. A method of diagnosing a form of lower back pain (LBP) in an individual, comprising:
obtaining a sample from the individual;
assaying the sample to determine the type and/or concentration of one or more inflammatory mediators in the individual; and
diagnosing the form of LBP based upon the type and/or concentration of one or more inflammatory mediators.

2. The method of claim 1, wherein the sample is obtained from the epidural space of the individual.

3. The method of claim 1, wherein the one or more inflammatory mediators comprise eicosanoids and/or cytokines.

4. The method of claim 1, wherein the one or more inflammatory mediators comprise prostaglandin E2 (PGE2), leokotriene B4 (LTB4), thromboxane A2 (TXA2), tumor necrosis factor alpha (TNFa), interleukin-1 (IL-1), interleukin-6 (IL-6), and/or nitric oxide (NO).

5. The method of claim 1, wherein sample is obtained from the individual by microdialysis.

6. The method of claim 1, wherein the sample is obtained from the individual using a CMA-71 High Cut-Off Brain Microdialysis Catheter.

7. The method of claim 1, wherein the sample is assayed using a multiplexed assay kit.

8. The method of claim 1, wherein the sample is assayed using ELISA and/or electrochemiluminescence.

9. The method of claim 1, wherein the sample is assayed by measuring citruline concentrations.

10. The method of claim 1, wherein the form of LBP comprises acute LBP, chronic LBP, clinical responsiveness to epidural steroids and/or responsiveness to drugs specifically targeting inflammatory mediators.

11. A method for treating a form of lower back pain (LBP) in an individual, comprising:
identifying a type and/or concentration of one or more inflammatory mediators in the epidural space of the individual;
rendering a diagnosis of the form of LBP in the individual based on the presence, absence and/or combination of presences and/or absences of one or more inflammatory mediators in the epidural space of the individual; and
treating the form of LBP.

12. The method of claim 11, wherein one of the one or more inflammatory mediators in the epidural space of the individual comprises acute LBP, chronic LBP, clinical responsiveness to epidural steroids and/or responsiveness to drugs specifically targeting inflammatory mediators.

13. A system for diagnosis of a form of lower back pain (LBP) in an individual, comprising:
a means to obtain a sample from the epidural space of the individual; and
an assay to identify a type and/or a concentration of one or more inflammatory mediators from the sample.

14. The system of claim 13, wherein the means to obtain a sample from the epidural space of the individual comprises microdialysis.

15. The system of claim 14, wherein the microdialysis uses a CMA-71 High Cut-Off Brain Microdialysis catheter.
